(19) United States
(12) Patent Application Publication
(10) Pub. No.: US 2026/0248902 A1
(43) Pub. Date: Aug. 27, 2026

(54) RECOMBINANT ADENOVIRUS COMPRISING A POLYNUCLEOTIDE ENCODING DENV ENVELOPE PROTEIN AND A POLYNUCLEOTIDE ENCODING FERRITIN HEAVY CHAIN PROTEIN, AND USES THEREOF

(71) Applicant: THE INDUSTRY & ACADEMIC COOPERATION IN CHUNGNAM NATIONAL UNIVERSITY (IAC), Daejeon (KR)

(72) Inventor: Hyun Jin SHIN, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 19/550,300

(22) Filed: Feb. 26, 2026

(30) Foreign Application Priority Data
Feb. 27, 2025 (KR) 10-2025-0026022

(51) Int. Cl. A61K 39/12; A61K 39/00; A61P 31/14; C07K 14/005; C07K 14/435; C12N 7/00

(52) U.S. Cl. CPC A61K 39/12; A61P 31/14; C07K 14/005; C07K 14/435; C12N 7/00; A61K 2039/5256; A61K 2039/54; A61K 2039/575; C07K 2319/00; C12N 2710/10022; C12N 2710/10043; C12N 2770/24122; C12N 2770/24134

(57) ABSTRACT

This disclosure relates to a recombinant adenovirus comprising a polynucleotide encoding DENV envelope protein and a polynucleotide encoding ferritin heavy chain protein, and uses thereof. This disclosure also provides a recombinant adenovirus comprising a polynucleotide encoding DENV (Dengue Virus) envelope protein and a polynucleotide encoding ferritin heavy chain protein, and a vaccine composition for preventing DENV infection comprising the recombinant adenovirus as an effective ingredient.

### CROSS-REFERENCE TO RELATED APPLICATION

This application is based on and claims priority under 35 U.S.C. § 119 to Korean Patent Application No. 10-2025-0026022, filed on Feb. 27, 2025, in the Korean Intellectual Property Office, the disclosure of which is incorporated by reference herein in its entirety.

### REFERENCE TO A SEQUENCE LISTING

The instant application contains a Sequencing Listing which has been submitted electronically in XMIL file and is hereby incorporated by reference in its entirety. Said XML copy, created on Feb. 9, 2026, is named 163437US-sequencing_listing and is 16,065 bytes in size.

### BACKGROUND

### 1. Field

This disclosure relates to a recombinant adenovirus comprising a polynucleotide encoding DENV envelope protein and a polynucleotide encoding ferritin heavy chain protein, and uses thereof.

### 2. Description of Related Art

Dengue Virus is a highly contagious flavivirus that is transmitted by certain species of mosquitoes and causes viral infection in humans. Dengue Virus exists as four serotypes, DENV-1, DENV-2, DENV-3, and DENV-4, and each serotype is classified according to diverse interaction between virus types and human immunity. According to reports from the WHO, it has been shown that the number of cases of dengue infection has increased over the last five years, and there remains needs for the development of vaccines and antiviral drugs to address this. Vaccines remain highly limited to be used against dengue virus, and further development of vaccines with enhanced immune responses and safety is required.

The genome of the dengue virus encodes three dominant structural proteins: the capsid, premembrane (prM), envelope, and seven non-structural proteins. Among these, structural proteins are important for viral entry process of viruses into host cells, and especially, envelope proteins are the major structural elements that interact with the host cell membrane. Currently, several vaccine strategies for the dengue virus are being investigated, including live attenuated vaccines, inactivated virus vaccines, recombinant subunit vaccines, viral vector vaccines, and DNA vaccines. In most vaccine candidates, the DENV envelope protein is used as the primary antigen. As viral vector-based vaccines, adenovirus, alphavirus, and vaccinia virus vectors have been used and studies for the cloning of antigenic molecules and delivery into host cells are being conducted. Vaccines using recombinant viruses induce durable immune responses, provide advantages in vaccine stability and large-scale manufacturing, and are effective in eliciting robust antibody responses and cellular immune responses.

Under such a technical background, the present inventors have generated, using an adenovirus vector system, a recombinant adenovirus comprising a polynucleotide encoding DENV envelope protein and a polynucleotide encoding human ferritin heavy chain protein by conjugating dengue virus envelope protein with the human ferritin heavy chain and have completed the present disclosure by confirming the excellent vaccine efficacy of the recombinant adenovirus against DENV infection.

### SUMMARY

One aspect provides a recombinant adenovirus comprising a polynucleotide encoding DENV (Dengue Virus) envelope protein and a polynucleotide encoding ferritin heavy chain protein.

Another aspect provides a vaccine composition for preventing DENV infection comprising the recombinant adenovirus as an effective ingredient.

Another aspect provides an isolated nucleic acid molecule comprising a polynucleotide consisting of a nucleotide sequence of SEQ ID NO: 8, which encodes DENV envelope protein, and a polynucleotide consisting of a nucleotide sequence of SEQ ID NO: 10, which encodes ferritin heavy chain, wherein the polynucleotide encoding DENV envelope protein and the polynucleotide encoding ferritin heavy chain are directly linked to each other or are linked to each other via a linker sequence.

Other purposes and advantages of the present disclosure will become more obvious with the following detailed description, claims, and drawings.

Contents not described in the present specification will be sufficiently recognized and inferred by those skilled in the technical field of the present disclosure or in a similar technical field therewith, and thus descriptions of such contents will be omitted. Additional aspects will be set forth in part in the description which follows and, in part, will be apparent from the description, or may be learned by practice of the presented embodiments of the disclosure.

Each of descriptions and embodiments disclosed in the present disclosure may be applied to any of the other descriptions and embodiments. In other words, all combinations of the various elements disclosed in the present disclosure fall within the scope of the present application. Furthermore, the scope of the present disclosure is not to be considered limited by the specific descriptions set forth below.

One aspect provides a recombinant adenovirus comprising a polynucleotide encoding DENV (Dengue Virus) envelope protein and a polynucleotide encoding ferritin heavy chain protein.

The term “envelope protein” as used in the present specification refers to a structural protein located in the viral envelope, which is present on the surface of the viral particle, plays a key role in the process of infection of a host cell, and mediates viral attachment and fusion with host cell membrane, thereby facilitating delivery of the viral genome into the host cell. The envelope protein has a structure and function that are specific to each type of virus. For example, in DENV, which is a member of the genus Flavivirus, the envelope protein is referred to as the E protein, and it binds to receptor on the host cell and mediates pH-dependent membrane fusion, thereby allowing the virus to infect the host cell.

In an embodiment, the polynucleotide encoding DENV envelope protein and the polynucleotide encoding ferritin heavy chain may be directly linked to each other or are linked to each other via a linker sequence.

The term “identity” as used in the present specification refers to the overall relevance between polymer molecules, such as, nucleic acid (e.g., DNA molecules and/or RNA molecules) and/or between polypeptides. For example, polypeptides are considered “substantially identical” when amino acid sequences thereof have at least 60%, 65%, 70%, 75%, 80%, 85%, 90%, 95%, or 99% identity. Calculating the percent identity of two nucleic acid or polypeptide sequences can be performed by, for example, aligning two sequences for optimal comparison purposes (e.g., a gap can be introduced in one or both of first and second sequences for the optimal alignment, and non-identical sequences can be ignored for the comparison purposes). For example, the length of a sequence aligned for the comparison purposes is at least 60%, at least 70%, at least 80%, at least 90%, at least 95%, or substantially 100% of the length of a reference sequence. Subsequently, the nucleic acid or polypeptide sequences at corresponding positions are then compared. Determination of the percent identity between the two sequences and comparison of these sequences may be accomplished by using mathematical algorithms. As is well known to those skilled in the art, the amino acid or nucleic acid sequences may be compared by using any of a variety of algorithms including those available in the commercial computer programs, specifically, BLASTN for nucleotide sequences, and BLASTP, gapped BLAST, or PSIBLAST for amino acid sequences.

In an embodiment, the polynucleotide encoding DENV envelope protein may consist of a nucleotide sequence of SEQ ID NO: 8. In addition, the polynucleotide encoding DENV envelope protein may not only consist of the nucleotide sequence of SEQ ID NO: 8, but also a nucleotide sequence having 80 to 99% identity with the sequence of SEQ ID NO: 8.

In an embodiment, the DENV envelope protein may comprise an amino acid sequence of SEQ ID NO: 12. In addition, the DENV envelope protein may not only comprise the amino acid sequence of SEQ ID NO: 12, but also an amino sequence having 80 to 99% identity with the sequence of SEQ ID NO: 12.

The term “ferritin” as used in the present specification refers to a protein that stores iron and is widely present in prokaryotes and eukaryotes. Ferritin has a molecular weight of about 500,000 Da, is composed of a heavy chain and a light chain, and exhibits the unique property of forming spherical particles due to its self-assembly ability. Ferritin is a protein in which 24 monomers (a single monomer or heterogeneous monomer composed of either a heavy chain or a light chain) are gathered to form a huge spherical tertiary structure. In the case of human ferritin, the outer diameter is about 12 nm, and the inner diameter is about 8 nm. Ferritin dissociates into monomers or forms nano particles composed of 24 assembled monomers, depending on pH.

In an embodiment, the polynucleotide encoding ferritin heavy chain protein may consist of a nucleotide sequence of SEQ ID NO: 10. In addition, the polynucleotide encoding ferritin heavy chain protein may not only comprise the nucleotide sequence of SEQ ID NO: 10, but also a nucleotide sequence having 80 to 99% identity with the sequence of SEQ ID NO: 10.

The ferritin heavy chain protein may be derived from a human or non-human ferritin protein and may comprise all or a part of the amino acid sequence of the heavy chain constituting the ferritin.

In an embodiment, the ferritin heavy chain protein may comprise an amino acid sequence of SEQ ID NO: 14. In addition, the ferritin heavy chain protein may not only comprise the amino acid sequence of SEQ ID NO: 14, but also an amino acid sequence having 80 to 99% identity with the sequence of SEQ ID NO: 14.

In an embodiment, the polynucleotide encoding DENV envelope protein may be linked at its 5′end to a polynucleotide encoding precursor membrane protein (prM), either directly or via a linker sequence.

The term “prM protein” as used in the present specification refers to an essential structural protein belonging to the family Flaviviridae, such as Dengue virus, which initially exists in an inactive form and is converted into an active form during viral particle maturation by cleavage at a specific site, and is a post-translationally modified protein.

This cleavage process of prM plays a critical role in converting viral particles from a non-infectious state to infectious state and serves as a key regulatory step in the viral life cycle.

The recombinant adenovirus may further comprise the polynucleotide encoding prM protein, thereby enhancing the expression efficiency of the target protein, that is, the DENV envelope protein and the ferritin heavy chain protein.

According to an embodiment, the polynucleotide encoding prM protein may be a polynucleotide encoding a part of the prM protein.

In an embodiment, the polynucleotide encoding prM protein may consist of a nucleotide sequence of SEQ ID: 7. In addition, the polynucleotide encoding prM protein may not only comprise the nucleotide sequence of SEQ ID NO: 7, but also a nucleotide sequence having 80 to 99% identity with the sequence of SEQ ID NO: 7.

In an embodiment, the prM protein may comprise an amino acid sequence of SEQ ID: 11. In addition, the prM protein may not only comprise the amino acid sequence of SEQ ID NO: 11, but also an amino acid sequence having 80 to 99% identity with the sequence of SEQ ID NO: 11.

In an embodiment, the polynucleotide encoding prM protein, the polynucleotide encoding DENV envelope protein, and the polynucleotide encoding ferritin heavy chain protein may be sequentially linked from the 5′ end, either directly or via a linker sequence.

The term “Recombinant adenovirus” as used in the present specification refers to a non-enveloped virus or virus vector with a linear double-stranded DNA genome (34-43 kb).

The recombinant adenovirus may be a genetic element for delivering a target gene, for example the polynucleotide encoding DENV envelope protein and the polynucleotide encoding ferritin heavy chain protein, to a target tissue or cell and for stably expressing the target gene. To this end, the recombinant adenovirus may further comprise, in addition to the target gene, i.e., the polynucleotide sequence encoding DENV envelope protein and the polynucleotide sequence encoding ferritin heavy chain protein, a nucleic acid construct comprising elements necessary for expression of the target gene/protein.

Specifically, the recombinant adenovirus may further comprise a nucleic acid construct comprising an expression regulatory sequence including at least a promoter and a nucleic acid for expressing the target protein, the nucleic acid being operably linked to the expression regulatory sequence.

For the purposes of the present disclosure, the target protein may be a protein in which DENV envelope protein and ferritin heavy chain protein are fused to each other or are operably linked to each other.

The term “expression regulatory sequence” as used in the present specification refers to a nucleic acid sequence essential for the expression of a coding sequence for a target protein to which it is operably linked in a particular host organism.

For example, it is known to use promoters, polyadenylation signals and enhancers as expression regulatory sequences suitable for eukaryotic cells.

The term “promoter” as used in the present specification refers to a nucleic acid fragment that regulates the transcription of one or more coding sequences and is located upstream, in the reading direction relative to the direction of transcription, from the transcription start site of the coding sequence, and that is structurally characterized by the presence of a binding site for a DNA-dependent RNA polymerase, a transcription start site, and additional nucleic acid sequences, including, but not limited to, transcription factor binding sites, repressor and activator protein binding sites, and other nucleotide sequences known to those skilled in the art that directly or indirectly regulate the transcription level of the promoter. As the promoter, any promoter that functions in mammalian cells may be exemplified, and examples thereof include constitutive promoters such as CMV promoter, SV40 promoter, EF-1α promoter, CAG promoter and PGK promoter, and U3 promoter, U6 promoter and H1 promoter and the like. In addition to these promoters, known inducible promoters, tissue- or organ-specific promoters, temporally specific promoters, or mutant sequences that are functionally equivalent thereto may be used in the present disclosure.

The term “enhancer” as used in the present specification refers to a nucleic acid sequence located adjacent to a sequence encoding a target protein. An enhancer element may be located 5′ upstream of a promoter element or may be located downstream of or within a coding nucleic acid sequence (e.g., a DNA sequence that is transcribed and/or translated into a recombinant product or products). That is, the enhancer element may be located 100, 200, or 300 base pairs (bp) upstream or downstream from the nucleic acid sequence encoding the target protein, or at a greater distance.

The term “operably linked” as used in the present specification refers to the linkage of nucleotide sequences on a single nucleic acid fragment such that the function of one is affected by the other.

In an embodiment, the recombinant adenovirus may comprise the nucleic acid construct shown in FIG. 1B, for example, a construct in which an envelope ferritin heavy chain (E-H) clone according to an embodiment is inserted to a pacAd5 CMV-N-pA vector, but is not limited thereto.

According to an embodiment, the recombinant adenovirus into which polynucleotides encoding DENV envelope protein and ferritin heavy chain protein were inserted was prepared, and it was confirmed that the recombinant adenovirus was capable of stably expressing, in target cells, the target proteins, namely DENV envelope protein and ferritin heavy chain protein.

In addition, the administration of the recombinant adenovirus was found to significantly enhance the IgG-mediated humoral immune response against a DENV antigen in a mouse model. Therefore, the recombinant adenovirus according to an aspect may be used as an effective ingredient in a vaccine composition for preventing DENV infection.

In one or more embodiments, the recombinant adenovirus is configured to deliver the polynucleotides encoding the DENV envelope protein and the ferritin heavy chain protein into cells of a subject. Upon administration, the expressed fusion proteins or protein complexes comprising the ferritin heavy chain are characterized by their ability to spontaneously self-assemble in vivo, thereby forming spherical nanoparticles. Such spherical nanoparticles present the DENV envelope protein as an antigen on their surface in a multivalent display, which can enhance recognition by immune cells and is capable of eliciting a robust immune response significantly superior to that of monomeric antigens.

Another aspect provides a vaccine composition for preventing DENV infection comprising the recombinant adenovirus as an effective ingredient; and a pharmaceutical use of the recombinant adenovirus for preventing DENV infection.

Since the vaccine composition and its pharmaceutical use include or use the above-described recombinant adenovirus, descriptions in common therebetween will be omitted to avoid undue complexity in the present specification.

The term “effective ingredient” as used in the present specification refers to an appropriate effective amount of an ingredient that brings about a beneficial or desirable clinical or biochemical outcome. Specifically, the term may refer to an agent, an active agent, or a recombinant virus of an effective amount. The effective amount may be administered one or more times, and unlimitedly may be an appropriate amount for preventing a disease, alleviating symptoms, reducing the extent of the disease, stabilizing (i.e., not exacerbating) the disease state, delaying or reducing the rate of disease progression, or improving or temporarily alleviating and ameliorating (partially or fully) the disease state.

The term “prevention” as used in the present specification refers to any action that blocks an occurrence of a disease in advance, suppresses a disease, or delays progression of a disease. For example, the term refers to preventing the DENV infection or occurrence of characteristics thereof, interrupting the occurrence, or defending or protecting from the DENV infection or occurrence of characteristics thereof.

The term “effective amount” as used in the present specification refers to a generally accepted meaning in the art. The term generally refers to an amount of a molecule, compound, or composition that elicits an intended biological response (e.g., a beneficial response) in a cell, tissue, a system, an animal, or a human, sought by researchers, veterinarians, physicians, or other clinicians, etc. Specifically, a “therapeutically effective amount” refers to an amount of a molecule, compound, or composition that is capable of eliciting a desirable medical response to the extent that a particular clinical treatment may be considered effective, due to, for example, a therapeutically relevant change in a measurable parameter associated with the disease or disorder. The therapeutically effective amount of a drug for treatment of a disease or a disorder may be an amount necessary to cause a therapeutically relevant change in the parameter.

In an embodiment, the present disclosure relates to a pharmaceutical composition/vaccine composition comprising the recombinant adenovirus of the present disclosure in a pharmaceutically acceptable carrier, another pharmaceutical material, an adjuvant, a diluent, or a combination thereof. The vaccine composition may be selected from the group consisting of compositions for intramuscular administration, subcutaneous administration, intraperitoneal administration, intravenous administration, dermal administration, ocular administration, and intracerebral administration. When the vaccine composition is administered by injection or systemically, the carrier may be typically a liquid carrier. For other methods of administration, the carrier may be a solid or a liquid, for example sterile pyrogen-free water or a sterile pyrogen-free phosphate-buffered saline solution. As an injectable medium, it is preferable to use water containing additives that are commonly used in injectable solutions, such as stabilizers, salts or saline, and/or buffers.

The vaccine composition refers to a composition comprising the recombinant adenovirus of the present disclosure and one or more component that are pharmaceutically acceptable and pharmacologically appropriate excipients selected from the group consisting of fillers, solvents, diluents, carriers, adjuvants, disintegrants, delivery agents, preservatives, stabilizers, emulsifiers, suspending agents, thickeners, and sustained-released modifiers, and the selection and ratio thereof are determined according to the type and route of administration and the dosage. The vaccine composition of the present disclosure and a method for preparing the same will be apparent to those skilled in the art without undue difficulty. The vaccine composition should preferably be prepared in accordance with Good Manufacturing Practice (GMP) requirements. The composition may also comprise a buffer composition, a tonicity agent, a stabilizer, and a solubilizer.

The vaccine composition may be prepared, packaged, or widely marketed in the form of a ready formulation as a single unit dose or as a plurality of single unit doses. The term “single unit dose” as used in the present specification refers to a discrete amount of the vaccine composition containing a predetermined quantity of the active ingredient. The amount of the active ingredient typically corresponds to a dosage of the active ingredient to be administered to a subject, or for convenience, to a fraction of such dosage, for example one-half or one-third of the dosage.

Another aspect provides a method for preventing, ameliorating or treating DENV infection, comprising administering the vaccine composition to a subject in need thereof.

Since the method for preventing, ameliorating or treating DENV infection includes or uses the above-described recombinant adenovirus or vaccine composition, descriptions in common therebetween will be omitted to avoid undue complexity in the present specification.

In an embodiment, the DENV infection may be at least one selected from the group consisting of Dengue Fever (DF), Dengue Hemorrhagic Fever (DHF), Dengue Shock Syndrome (DSS), and severe dengue.

The term “subject” as used in the present specification refers to a subject in need of prevention or treatment for a disease, specifically, DENV infection, and more specifically, the term may include all mammals such as a human or a non-human primate, a mouse, a dog, a cat, a horse, a cow, sheep, a pig, a goat, a camel, and an antelope.

Another aspect provides an isolated nucleic acid molecule comprising a polynucleotide consisting of a nucleotide sequence of SEQ ID NO: 8, which encodes DENV envelope protein, and a polynucleotide consisting of a nucleotide sequence of SEQ ID NO: 10, which encodes ferritin heavy chain protein, wherein the polynucleotide encoding DENV envelope protein and the polynucleotide encoding ferritin heavy chain are directly linked to each other or are linked to each other via a linker sequence.

In an embodiment, the polynucleotide consisting of the nucleotide sequence of SEQ ID NO: 8, which encodes DENV envelope protein, may be linked at its 5′ end to a polynucleotide consisting of a nucleotide sequence of SEQ ID NO: 7, which encodes precursor membrane protein (prM), either directly or via a linker sequence.

In an embodiment, the polynucleotide consisting of the nucleotide sequence of SEQ ID NO: 7, which encodes precursor membrane protein (prM), the polynucleotide consisting of the nucleotide sequence of SEQ ID NO: 8, which encodes DENV envelope protein, and the polynucleotide consisting of the nucleotide sequence of SEQ ID NO: 10, which encodes ferritin heavy chain protein, may be sequentially linked from the 5′ end, either directly or via a linker sequence.

Since the isolated nucleic acid molecule shares its technical constitution with the above-described recombinant adenovirus or vaccine composition, descriptions in common therebetween will be omitted to avoid undue complexity in the present specification.

The term “nucleic acid” as used in the present specification refers to a polymeric material comprising a plurality of nucleotide units and, more specifically, to a polymer in which a plurality of nucleotide units are linked to one another through phosphodiester bonds of a sugar-phosphate backbone. The nucleic acid may be used interchangeably with the term, “polynucleotide” and “nucleic acid molecule”. The nucleic acid is an essential biopolymer in living organisms and may be RNA or DNA, preferably DNA, which encodes genetic information through its specific nucleotide sequence. The nucleic acid may be isolated, artificially synthesized, or non-naturally occurring or engineered, wherein the term “non-naturally occurring or engineered” refers to a state in which the nucleic acid does not exist in its natural form but is produced by artificial modification. Here, the artificial modification is intended to enhance expression of the target protein and may comprise codon-optimized coding sequences of the DENV envelope protein and/or the ferritin heavy chain protein.

The term “isolated” nucleic acid (e.g., “isolated DNA”) as used in the present specification refers to a polynucleotide that is at least partially separated from at least some of the other components of a naturally occurring organism or virus, e.g., cellular or viral structural components, other polypeptides, or nucleic acids that are ordinarily found associated with the polynucleotide.

In an embodiment, the isolated nucleic acid molecule may be inserted into a viral vector (e.g., an adenoviral vector) and administered to a subject.

In an embodiment, the isolated nucleic acid molecule may be delivered to a subject via the viral vector, thereby inducing an immune response against a DENV antigen. Here, the immune response may include an IgG-mediated humoral immune response.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a vector map of a packaging vector used to generate a recombinant adenovirus according to an embodiment.

FIG. 1B is a vector map of a shuttle vector used to generate a recombinant adenovirus according to an embodiment.

FIG. 1C shows a result of confirming the expression levels of the envelope (E) and the envelope ferritin heavy chain (E-H) protein as determined by western blotting using an anti-Myc antibody after transfection with a recombinant plasmid according to an embodiment.

FIG. 1D shows a result of confirming the expression levels of the envelope (E) protein and the envelope-ferritin heavy chain (E-H) protein, as determined by western blotting using an anti-ferritin heavy chain antibody after transfection with a recombinant plasmid according to an embodiment.

FIG. 2A shows a result of confirming the cytopathic effect (CPE) specific to a recombinant adenovirus according to an embodiment.

FIG. 2B shows a result of confirming the presence or absence of the envelope (E) protein and the envelope-ferritin heavy chain (E-H) genes from DNA derived from a culture supernatant of a recombinant adenovirus according to an embodiment.

FIG. 2C shows a result of confirming the presence or absence of an adenovirus band in a recombinant adenovirus according to an embodiment.

FIG. 2D shows a result of performing a TCID50 assay to analyze the titer of a recombinant adenovirus according to an embodiment.

FIG. 3A shows a result of confirming the expression levels of the envelope (E) and the envelope ferritin heavy chain (E-H) protein in cells transduced with a recombinant adenovirus according to an embodiment, as determined by western blotting using an anti-Myc antibody.

FIG. 3B shows a result of confirming the expression levels of the envelope (E) and the envelope ferritin heavy chain (E-H) protein in cells transduced with a recombinant adenovirus according to an embodiment, as determined by western blotting using an anti-ferritin heavy chain antibody.

FIG. 4A schematically shows an experimental procedure using a mouse animal model to evaluate an immune response of a vaccine composition comprising a recombinant adenovirus according to an embodiment.

FIG. 4B shows a result of confirming, by an enzyme-linked immunosorbent assay (ELISA), a humoral immune response induced by administration of a vaccine composition comprising a recombinant adenovirus according to an embodiment.

FIG. 4C shows a result of comparing humoral immune responses at 1 week and 4 weeks post-vaccination, induced by administration of a vaccine composition comprising a recombinant adenovirus according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the disclosure will be described in detail with reference to Examples below. However, these Examples are for illustrative purposes only, and the scope of the disclosure is not intended to be limited by these Examples.

### EXPERIMENTAL EXAMPLES

### Experimental Example 1. Preparation of Cells and Viruses

Vero cells were maintained in Minimum Essential medium (MEM)(WELGENE, Korea) supplemented with 10% fetal bovine serum (FBS) (Biowest, Lakewood Ranch, FL, USA), 1% antibiotics-antimycotics (AA) (Biowest, Lakewood Ranch, FL, USA) and 1% HEPES (Gibco, USA). HEK 293T and 293A cells were maintained in Dulbecco's modified Eagle's medium (DMEM)(Biowest, Lakewood Ranch, FL, USA) containing 10% FBS, 1% AA and 1% HEPES. A549 cells were maintained in RPMI 1640 medium (WELGENE, Korea) supplemented with 10% FBS and 1% AA. The DENV-2/KBPV-VR-29 strain was used in this study.

### Experimental Example 2. Virus Culture

DENV2 was inoculated into Vero cells in serum-free MEM in the presence of 2% FBS and 1% AA and incubated for 3-5 days in a CO₂ incubator at 37° C.

The virus culture was maintained for 4-5 passages in the same manner, increasing the scale. Finally, the virus was harvested and centrifuged at 12,000×g for 10 min at 4° C. to recover the supernatant. The cell pellet was suspended in virus stock solution, lysed with three freeze-thaw cycles, frozen in liquid nitrogen (LN2), and thawed in a 37° cwater bath to release intracellular virus particles. The cells were then centrifuged at 12,000×g for 10 min to pellet cell debris, and the virus-containing supernatant was mixed with the virus supernatant. The viruses were purified and concentrated via centrifugation using a sucrose gradient. Briefly, 25 mL of virus supernatant was carefully decanted into 10 mL of 20% sucrose dissolved in phosphate-buffered saline (PBS) and ultra-centrifuged in a Hitachi CP100NX ultracentrifuge at 72,000×g for 5 h at 4° C. The supernatant was aspirated and 200 μL of PBS containing 1% FBS was added to the pellet, followed by incubation at 4° C. overnight. The next day, the virus pellet suspended in the solution was aliquoted into microcentrifuge tubes and stored at −80° C.

### Experimental Example 3. Viral RNA Isolation, cDNA Preparation, and qPCR

Viral RNA was isolated from 300 μL of virus supernatant using Ribospin™ vRD (GeneAll, Seoul, Republic of Korea) and cDNA was prepared using TOPscript™RT DryMIX (dN18/dN6) (Enzynomics, Daejeon, Republic of Korea). To confirm the cultured and purified viruses, 1 μL of cDNA was used for qPCR analysis with 1 μL of each previously reported primer; Primer 1, Primer 2 (Table 1), and 10 μL of 2× Real-Time PCR Master Mix (BioFACT™, Daejeon Republic of Korea) were added to the reaction mixture to a final volume of 20 μL. RT-qPCR was performed using a QuantGene 9600 Real Time Thermal Cycler (Bioer, Binjiang, China), with a standard two-step thermal cycling profile. The initial denaturation was performed at 95° C. for 5 min, then, denaturation at 95° C. for 10 s, and annealing and extension at 60° C. for 20 s, with 40 cycles of repeated denaturation and annealing. Relative expression levels were calculated using cycle threshold (Ct) values.

TABLE 1

| | | SEQ ID |
|---|---|---|
| Name | Sequence (5′→3′) | NO. |
| Primer 1 | CAAACAGCAGGACCTTGG | 1 |
| Primer 2 | ATCCATCCTCACCTCTGT | 2 |
| Primer 3 | GGTACCCTCGAGATGGACTACAAAGACGATGACGACAA | 3 |
| | GGCAGCAATCCTGGCATAC | |
| Primer 4 | GGAATTCCAGATCCTCTTCTGAGATGAGTTTTTGTTCACG | 4 |
| | CGTTCCTTTCTTGAACCAGTT | |
| Primer 5 | GGAATTCAGCAGCGGCACGACCGCGTCCACCTCGCA | 5 |
| Primer 6 | CGTCTAGATTAGCTTTCATTATCACTGTCTC | 6 |

### Experimental Example 4. Plasmids Construction

To generate rAds for DENV2 virus envelope (E) and envelope ferritin heavy chain genes (E-H), the RAPAd® CMV Adenoviral expression system (Cell Biolabs, San Diego, CA, USA) was used. pacAd5 CMV K-N pA (shuttle vector) and pacAd5 9.2-100 (packaging vector) were used and Each E and E-H gene was cloned into the multi-cloning sites of the shuttle vector. Initially, 93 nucleotides (31 amino acids) from the C-terminus of the prM gene and 1185 nucleotides (395 amino acids) from the N-terminus of the envelope gene were amplified from the prepared cDNA using Primer 3 and Primer 4 (Table 1), and then, was inserted in between enzyme sites; XhoI and EcoRI in pacAd5 CMV K-N pA vector and obtained pAd E plasmids. To construct E-H plasmid, the ferritin heavy chain gene which was amplified from cDNA isolated from 293T cells using Primer 5 protruding a linker (SSG) at the 5′ end, and Primer 6 was cloned in between EcoRI and XbaI enzyme sites downstream of prM31 envelope gene and obtained the pAd E-H plasmid.

### Experimental Example 5. Transfection of Recombinant Plasmids

The day before transfection, HEK 293T cells were cultured in six-well cell culture plates at a concentration of 1×10⁶ cells/well, and when the cells achieved 80-90% confluence, 200 μL of transfection mixture containing 3 μg of plasmid DNA and 9 μL of Polyethylenimine (PEI) was added to the cells. Subsequently, the medium was replaced, and the cells were incubated at 37° C. in a CO₂ incubator for 48 h and harvested for expression analysis.

### Experimental Example 6. Adenovirus Production, Amplification and Purification

For the generation of adenoviruses, 2.4 μg of shuttle vector containing the genes of interest, and 1.2 μg of Ad backbone vector were digested with PacI enzyme for vector linearization. One day before transfection, 2×10⁶ cells were seeded in a 60 mm-cell culture dish without antibiotics. When the cells achieved 70-80% confluence, the shuttle vector and backbone vector were co-transfected with Lipofectamine® 3000 transfection reagent (ThermoFisher Scientific, Waltham, MA, USA) according to the manufacturer's instructions. The next day, the medium was aspirated and 4 mL of fresh culture medium (DMEM supplemented with 10% FBS and 1% ampicillin) was added. Following incubation in a CO₂ incubator at 37° C. for 7-14 days, plaques were checked intermittently. The rAds generated from pAd E and pAd E-H were named rAd E and rAd E-H, respectively.

When appropriate cytopathic effect (CPE)/plaques appeared (50% of the cells were lifted), the cells and viral supernatant were harvested and centrifuged at 2000 rpm for 10 min. The cell pellet was suspended in the virus stock solution, and three freeze-thaw cycles were performed to release the intracellular virus, followed by centrifuging at 12,000 rpm for 10 min. Then, the supernatant was mixed with the previous supernatant, inoculated into a new 10 cm dish, and incubated in a CO₂ incubator at 37° C. for 1-2 h. Thereafter, the medium was replaced with 10 mL DMEM containing 5% FBS and 1% ampicillin and subsequently kept in an incubator for 2-3 days until all cells were detached. After repeating this inoculation procedure, 100 mL of the virus supernatant was obtained.

The virus supernatant was mixed with 50 mL of 20% PEG in a 2.5 mM NaCl solution. It was then incubated overnight in an inverter at 4° C. to precipitate the virus. The precipitated virus pellet was harvested after centrifugation at 7500 rpm for 30 min, and the supernatant was discarded. The pellet was resuspended in 2 mL 20 mM Tris-HCl (pH 8.0) and centrifuged at 12,000 rpm for 10 min to remove debris and recover the adenovirus-containing supernatant. For density gradient ultracentrifugation, CsCl solutions with densities of 1.3 g/mL and 1.4 g/mL were prepared in 20 mM Tris-HCL (pH 8). Sequentially, 1.5 mL of 1.4 g/mL solution, 1 mL of 1.3 g/mL solution, and 2 mL of the recovered adenovirus supernatant were added to the ultracentrifuge tube without disturbing each layer. Finally, the mixture was centrifuged at 100,000×g for 20 h at 4° C.

The obtained ultra-viral band was retrieved and inserted into a dialysis bag (molecular weight cutoff of 10,000) and dialyzed against 10 mM Tris-HCl (pH 8.0) overnight. Viruses were filtered through a 0.2 μM filter, aliquoted, and stored at −80° C. To determine the titer of rAds, a TCID50 assay was performed in 293A cells, and the TCID50 values were converted into PFU/mL.

### Experimental Example 7. Virus Transduction and Expression Check

One day before transduction, A549 cells (1.5×10⁶ cells/well) were seeded in six-well cell culture plates, followed by transduction of purified viruses into cells at different MOIs (2.5, 25, and 250) and incubated in a CO₂ incubator at 37° C. Cells were harvested 48 h post-transduction and protein expression was assessed by western blotting.

### Experimental Example 8. Western Blotting

Cells were lysed using Ottimolyse Ilysis buffer (Jubiotech, Daejeon, Republic of Korea), and similar amounts of protein samples prepared in the sample buffer were boiled and subjected to sodium dodecyl sulfate-polyacrylamide gel electrophoresis (SDS-PAGE). The separated proteins were transferred to a PVDF membrane (MERCK, Darmstadt, Germany), blocked with 5% skimmed milk, and treated with anti-Myc antibody (CSB-PA000085, Cusabio, Wuhan, China) or ferritin heavy chain antibody (GT1149, Gene Tex, Hsinchu, Taiwan) and an anti-rabbit secondary antibody (PA489724 Cusabio, Wuhan, China). The antibody-treated membrane was developed using a western blot enhancer and the images were analyzed using a luminograph.

### Experimental Example 9. Mouse Immunization

Six-week-old Female BALB/c mice were maintained at the animal facility of Chungnam National University (CNU), Republic of Korea, in accordance with the guidelines of the Institutional Animal Care and Use Committee. Mice were divided into three groups, with five mice in each group. rAds (8×10⁶ PFU/mL) were prepared in PBS without adjuvant. Mice in each group were immunized intramuscularly with 100 μL of rAd E, rAd E-H, or PBS. Mice were immunized twice at 2-week intervals followed by blood being collected by retro-orbital bleeding at 1 and 4 weeks after the second booster.

### Experimental Example 10. Evaluation of Humoral Immune Response (Enzyme-Linked Immunosorbent Assay: ELISA)

A hundred-fold diluted (10-1) dengue virus in PBS was added to each well of the immunoplates (100 pIwell) and incubated at 4° C. overnight. The next day, the plate was washed with PBS two times and blocked with 3% skimmed milk in PBS (blocking buffer) for 1 h. After additional washing with PBS, the plates were incubated with diluted serum samples (100 μpI/well) in the blocking buffer for 2 h. A washing buffer was prepared using 1% Triton X-100 in PBS and the plates were washed three times. The plates were then incubated with HRP-conjugated anti-mouse IgG (CSB-PA644737, Cusabio, Wuhan, China) diluted (1:10,000) in blocking buffer for 1 h. After washing three times with PBS-T, 100 μL of TMB substrate and stop solution were added sequentially. The absorbance values were recorded at 450 nm using a plate reader (PerkinElmer, Hopkinton, MA, USA).

### Examples

### Example 1. Generation of Recombinant Adenoviruses (rAds) Expressing Envelope Ferritin Heavy Chain (E-H) Protein

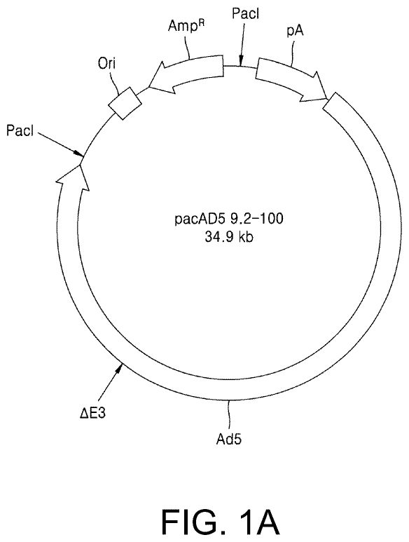

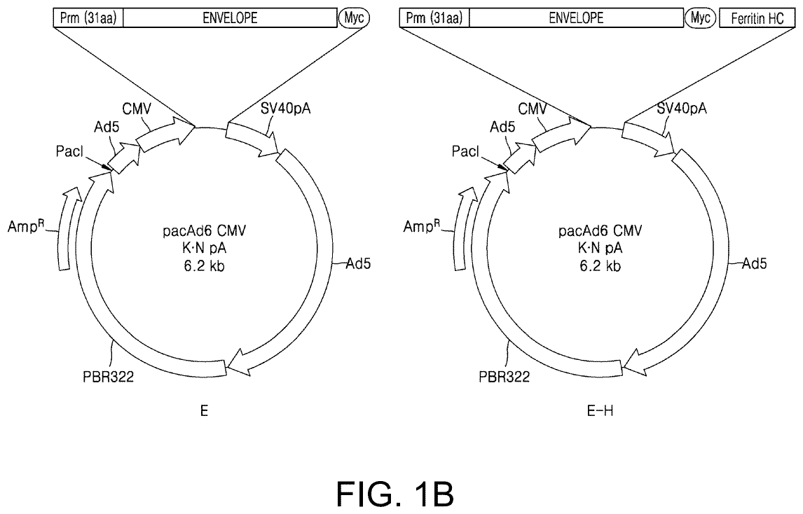

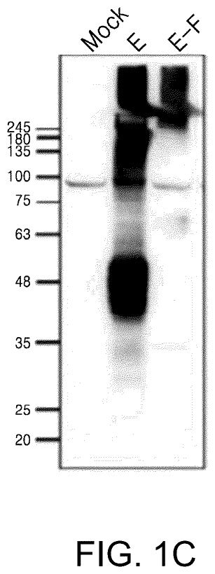

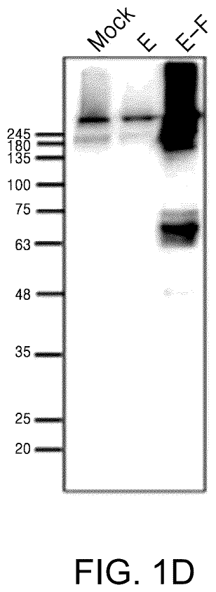

A recombinant adenovirus (rAds) expressing the E-H protein according to an embodiment was generated in the manner described in Experimental Examples 4 to 6. FIG. 1A is a vector map of a packaging vector used to produce the recombinant adenovirus (rAds) according to an embodiment, and FIG. 1B is a vector map of a shuttle vector used to produce the recombinant adenovirus (rAds) according to an embodiment.

The sequence of polynucleotide inserted into the adenovirus according to an embodiment was further confirmed (Cosmogenetech Co., Ltd), and the specific sequence information is provided below.

TABLE 2
Sequence SEQ ID

| Name | | NO. |
|---|---|---|
| prM nucleotide | ATGGCAGCAATCCTGGCATACACCATAGGAACGAC | 7 |
| (93 bp) | ACATTTCCAAAGAGTCCTGATTTTCATCCTACTGAC | |
| | AGCTGTCGCTCCTTCAATGACA | |
| DENV | ATGCGCTGTATAGGAATAGCAAATAGGGACTTCGT | 8 |
| envelope | GGAAGGGGTTTCAGGAGGAAGCTGGGTTGACATAG | |
| ectodomain (79% | TCTTAGAACACGGAAGCTGTGTGACGACGATGGCA | |
| C-term | AAAAACAAACCAACACTGGACTTTGAACTGATAAA | |
| deleted | AACAGAAGCCAAACAACCCGCCACTCTAAGGAAGT | |
| nucleotide) | ACTGCATAGAGGCTAAGCTGACCAACACGACAACA | |
| | GATTCGCGCTGCCCAACACAAGGGGAACCTAGCCT | |
| | GAATGAAGAGCAGGACAAAAGGTTTGTCTGCAAAC | |
| | ATTCTATGGTAGACAGAGGATGGGGAAATGGATGC | |
| | GGATTGTTTGGAAAAGGAGGCATCGTGACCTGTGC | |
| | TATGTTCACATGCAAAAAGAATATGAAAGGAAAAA | |
| | TTGTGCAGCCAGAAAACTTGGAATACACCATCGTG | |
| | ATAACACCTCACTCAGGGGAAGAACATGCAGTCGG | |
| | AAATAACACAGGAAAACATGGCAAGGAAGTCAAA | |
| | ATAACACCACAGAGCTCCATCACAGAAGCGGAACT | |
| | GACAGGCTATGGCACTGTTACGATGGAGTGCTCTCC | |
| | GAGAACGGGCCTCGACTTCAATGAGATGGTGTTGC | |
| | TGCAAATGGAAGATAAAGCTTGGCTGGTGCACAGA | |
| | CAATGGTTCCTAGACCTGCCGTTACCATGGCTGCCC | |
| | GGAGCAGATACACAAGGATCAAATTGGATACAGAA | |
| | AGAGACATTGGTCACTTTCAAAAATCCCCATGCAA | |
| | AAAAACAGGATGTTGTTGTCTTAGGATCCCAAGAG | |
| | GGGGCCATGCACACAGCACTCACAGGGGCTACGGA | |
| | AATCCAGATGTCATCAGGAAACCTACTGTTCACAG | |
| | GTCATCTTAAGTGCAGGCTGAGAATGGACAAACTA | |
| | CAGCTTAAAGGGATGTCATACTCTATGTGTACAGG | |
| | GAAGTTTAAAGTCGTGAAGGAAATAGCAGAAACAC | |
| | AACATGGAACAATAGTCATTAGAGTACAATATGAA | |
| | GGAGACGGCTCTCCATGCAAGATCCCTTTTGAGATA | |
| | ATGGATCTGGAAAAAAGACATGTCTTAGGTCGTCT | |
| | GATTACAGTCAACCCAATTGTAACAGAAAAGGACA | |
| | GGCCAGTCAACATAGAAGCAGAACCTCCATTCGGA | |
| | GACAGCTACATTATCATAGGAGTGGAGCCGGGACA | |
| | ACTGAAGCTTAACTGGTTCAAGAAAGGA | |
| Myc Tag | GAACAAAAACTCATCTCAGAAGAGGATCTG | 9 |
| nucleotide | | |
| Ferritin heavy | ATGACGACCGCGTCCACCTCGCAGGTGCGCCAGAA | 10 |
| chain | CTACCACCAGGACTCAGAGGCCGCCATCAACCGCC | |
| nucleotide | AGATCAACCTGGAGCTCTACGCCTCCTACGTTTACC | |
| | TGTCCATGTCTTACTACTTTGACCGCGATGATGTGG | |
| | CTTTGAAGAACTTTGCCAAATACTTTCTTCACCAAT | |
| | CTCATGAGGAGAGGGAACATGCTGAGAAACTGATG | |
| | AAGCTGCAGAACCAACGAGGTGGCCGAATCTTCCT | |
| | TCAGGATATCAAGAAACCAGACTGTGATGACTGGG | |
| | AGAGCGGGCTGAATGCAATGGAGTGTGCATTACAT | |
| | TTGGAAAAAAATGTGAATCAGTCACTACTGGAACT | |
| | GCACAAACTGGCCACTGACAAAAATGACCCCCATT | |
| | TGTGTGACTTCATTGAGACACATTACCTGAATGAGC | |
| | AGGTGAAAGCCATCAAAGAATTGGGTGACCACGTG | |
| | ACCAACTTGCGCAAGATGGGAGCGCCCGAATCTGG | |
| | CTTGGCGGAATATCTCTTTGACAAGCACACCCTGGG | |
| | AGACAGTGATAATGAAAGC | |
| prM amino | MAAILAYTIGTTHFQRVLIFILLTAVAPSMT | 11 |
| acid (31 amino | | |
| acids) | | |
| DENV | MRCIGIANRDFVEGVSGGSWVDIVLEHGSCVTTMAK | 12 |
| envelope | NKPTLDFELIKTEAKQPATLRKYCIEAKLTNTTTDSRC | |
| ectodomain (79% | PTQGEPSLNEEQDKRFVCKHSMVDRGWGNGCGLFGK | |
| C-term | GGIVTCAMFTCKKNMKGKIVQPENLEYTIVITPHSGEE | |
| deleted amino | HAVGNNTGKHGKEVKITPQSSITEAELTGYGTVTMEC | |
| acid) | SPRTGLDFNEMVLLQMEDKAWLVHRQWFLDLPLPW | |
| | LPGADTQGSNWIQKETLVTFKNPHAKKQDVVVLGSQ | |
| | EGAMHTALTGATEIQMSSGNLLFTGHLKCRLRMDKL | |
| | QLKGMSYSMCTGKFKVVKEIAETQHGTIVIRVQYEGD | |
| | GSPCKIPFEIMDLEKRHVLGRLITVNPIVTEKDRPVNIE | |
| | AEPPFGDSYIIIGVEPGQLKLNWFKKG | |
| Myc Tag | EQKLISEEDL | 13 |
| amino acid | | |
| Ferritin heavy | MTTASTSQVRQNYHQDSEAAINRQINLELYASYVYLS | 14 |
| chain amino | MSYYFDRDDVALKNFAKYFLHQSHEEREHAEKLMKL | |
| acid | QNQRGGRIFLQDIKKPDCDDWESGLNAMECALHLEK | |
| | NVNQSLLELHKLATDKNDPHLCDFIETHYLNEQVKAI | |
| | KELGDHVTNLRKMGAPESGLAEYLFDKHTLGDSDNE | |
| | S | |

### Example 2. Expression Evaluation of the E and the E-H Protein

In this example, using pAd E plasmid and pAd E-H plasmid generated in Example 1, HEK293 cells were transiently transfected in the manner described in Experimental Example 5, and then western blotting was performed in the manner described in Experimental Example 8 to evaluate the expression levels of the E protein and the E-H protein.

As a result, as shown in FIG. 1C, when an anti-Myc antibody was used, the recombinant E protein (49 kDa) was detected, whereas the expression level of the recombinant E-H protein was relatively low. Meanwhile, as shown in FIG. 1D, when an anti-ferritin heavy chain antibody was used, the recombinant E-H protein (68 kDa) was detected at a significant expression level. From these results, it was confirmed that the E-H protein can be stably expressed in cells following transfection with the recombinant adenovirus according to an embodiment.

### Example 3. rAds Purification, and Verification

In this Example, rAds generated in Example 1 were purified and verified. Before transfection of adenovirus-specific plasmids into 293A cells, plasmid DNA digested with the PacI enzyme was confirmed by running an agarose gel with non-digested DNA.

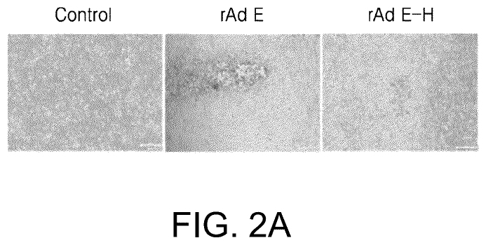

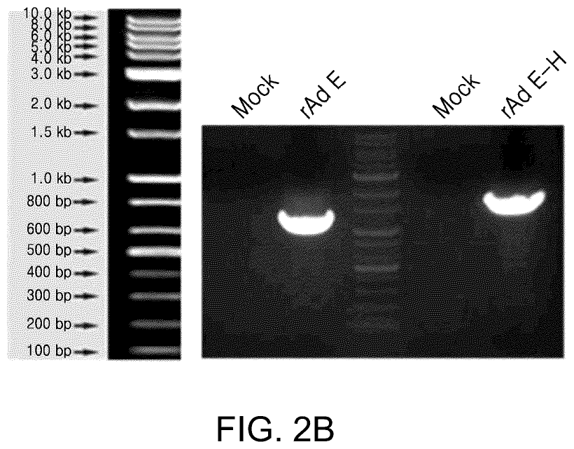

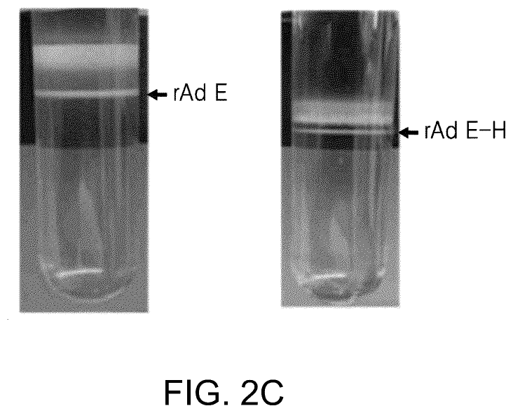

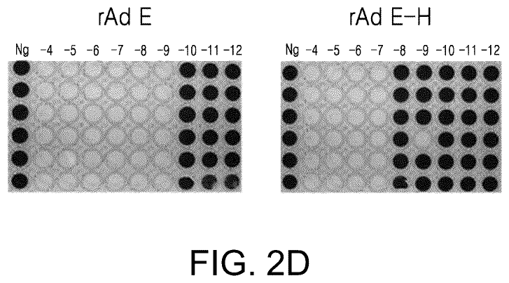

As shown in FIG. 2A, a specific cytopathic effect (CPE) of rAds were observed after 12 days of transfection with both pAdE and pAdE-H (FIG. 2A). The virus replication rate was low at the first passage stage, and from the third passage, it showed significant CPE within 3 days.

To confirm the presence of recombinant genes inside the virus, viral DNA was isolated, and PCR amplification was performed using the respective primers, and identified each DNA bands for recombinant E gene. As shown in FIG. 2B, E-H genes were confirmed using agarose gel electrophoresis and there were no bands in the negative control. As shown in FIG. 2C, after the virus was separated from the culture supernatant, clear adenoviral bands were obtained for rAd E and rAd E-H, and unpackaged viruses were observed over the clear packaged virus band.

Optimal buffer conditions were provided for adenoviruses, using dialysis and filtration to eliminate contamination. As shown in FIG. 2D, a clear CPE was observed after viral titration in 293A cells. The titers for rAd E and rAd E-H were calculated and they were 4.64×10¹⁰ TCID50/mL and 3.16×10⁸ TCID50/mL respectively. After converting these TCID50 values into PFU, it was obtained 2.60×10¹⁰ PFU/mL and 1.77×10⁸ PFU/mL respectively.

### Example 4. Evaluation of rAds Transduction Efficiency

In this Example, using the recombinant adenoviruses generated in Example 1, after the transduction of adenovirus into A549 cells following the manner of the Experimental Example 7, the in vitro generation of the recombinant E and the E-H proteins was evaluated performing western blotting in the manner of the Experimental Example 8.

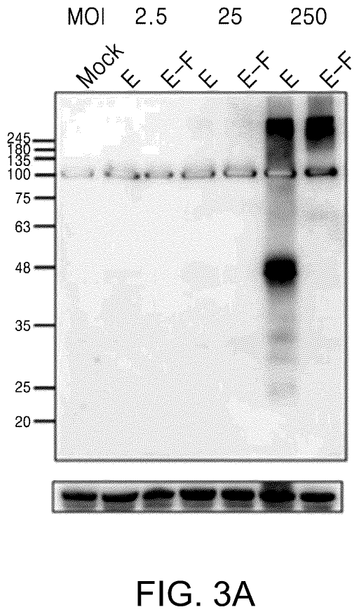

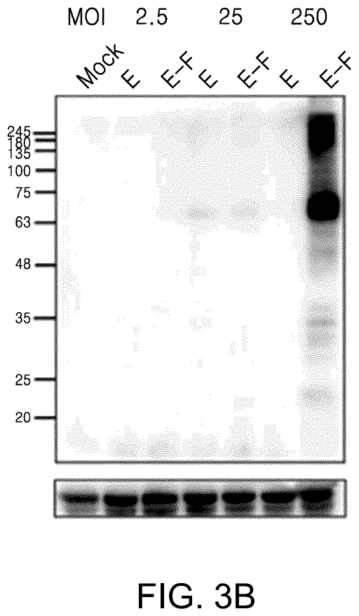

As a result, as shown in FIGS. 3A and 3B, respectively to the transduced MOI values the expression levels of proteins were also significantly differentiated and the pattern of protein expression was also the same as the transfection expression. Specifically, as shown in FIG. 3A, the recombinant E protein was detected at the correct level, whereas the E-H protein was detected very slightly using the anti-Myc antibody. Meanwhile, as shown in FIG. 3B, the expected size band of the recombinant E-H was detected using the ferritin heavy chain antibody, similar to the transfection expression data. Neither the E nor E-H proteins were detected with low MOI values (2.5 and 25, respectively), whereas 250 MOI transduction induced significant expression of the two proteins. From these results, it was confirmed that the E and E-H proteins can be stably expressed in cells following transduction with the recombinant adenovirus according to an embodiment at a high MOI (250).

### Example 5. Evaluation of Humoral Immune Response Using a Mouse Animal Model

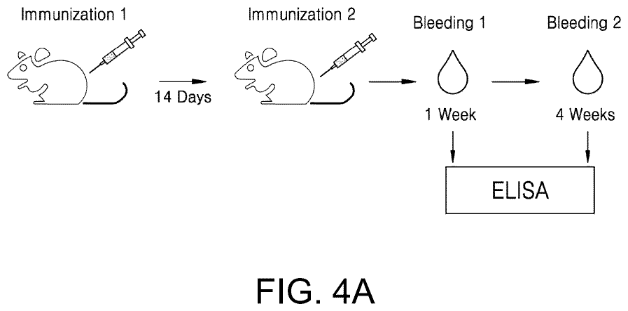

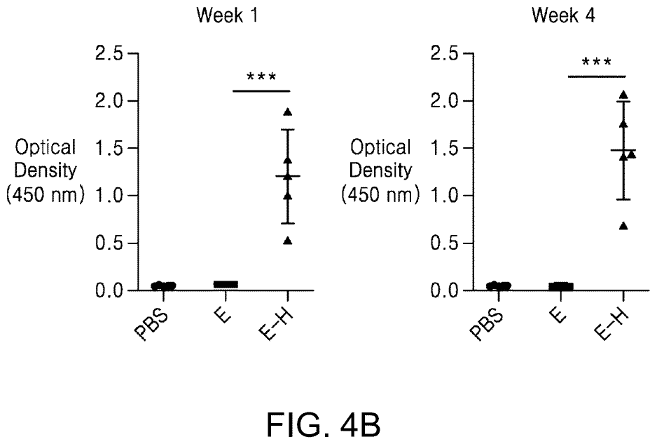

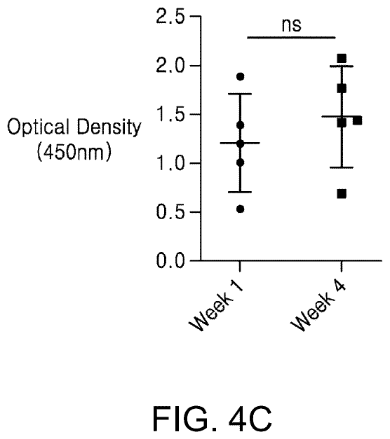

In this example, mice were immunized with the recombinant adenovirus (E-H rAdV) expressing the E-H protein, and the effect on the humoral immune response against DENV infection was evaluated. As shown in FIG. 4A, in the manner of the Experimental Example 9, BALB/c mice group (n=5) were immunized intramuscularly at 2-week intervals. Subsequently, in the manner of the Experimental Example 10, the humoral immune response for each mice group was confirmed by the Enzyme-Linked Immunosorbent Assay (ELISA). The cultured and purified DENV2 virus strain was confirmed by qPCR analysis of the serially diluted virus samples and the virus was used as a coating antigen for virus-specific ELISA.

As a result, as shown in FIG. 4B, rAd E-H induced a significant virus-specific IgG titer (p=0.0009) compared to rAd E in BALB/c mice at 1 week after the final immunization.

Similarly, as shown in FIG. 4B, even 4 weeks after immunization, rAd E-H showed a significant IgG response (p=0.0003) compared to the rAd E. Furthermore, as shown in FIG. 4C, the immune responses of the E-H immunized group were compared at 1 week and 4 weeks post-immunization; as a result, the IgG titers were maintained at a high level up to 4 weeks after immunization. From these results, it was confirmed that the vaccine composition according to an embodiment may significantly induce the IgG-mediated humoral immune response.

According to an embodiment, the vaccine composition may enhance an immune response against a DENV antigen by comprising, as an effective ingredient, a recombinant adenovirus comprising a polynucleotide encoding a DENV envelope protein and a ferritin heavy chain protein. Therefore, a polynucleotide according to an aspect or a recombinant adenovirus comprising the same may be used as an effective ingredient of a vaccine composition for preventing DENV infections.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A recombinant adenovirus comprising a polynucleotide encoding DENV (Dengue Virus) envelope protein and a polynucleotide encoding ferritin heavy chain protein.

2. The recombinant adenovirus of claim 1, wherein the polynucleotide encoding the DENV envelope protein and the polynucleotide encoding the ferritin heavy chain protein are directly linked to each other or are linked to each other via a linker sequence.

3. The recombinant adenovirus of claim 1, wherein the polynucleotide encoding the DENV envelope protein is linked at its 5′end to a polynucleotide encoding the precursor membrane protein (prM), either directly or via a linker sequence.

4. The recombinant adenovirus of claim 1, wherein the polynucleotide encoding the DENV envelope protein consists of a nucleotide sequence of SEQ ID NO: 8.

5. The recombinant adenovirus of claim 1, wherein the polynucleotide encoding the ferritin heavy chain protein consists of a nucleotide sequence of SEQ ID NO: 10.

6. The recombinant adenovirus of claim 3, wherein the polynucleotide encoding the prM protein consists of a nucleotide sequence of SEQ ID NO: 7.

7. A vaccine composition for preventing DENV infection, comprising the recombinant adenovirus of claim 1 as an effective ingredient.

8. The vaccine composition of claim 7, wherein the vaccine composition is selected from the group consisting of compositions for intramuscular administration, subcutaneous administration, intraperitoneal administration, intravenous administration, dermal administration, ocular administration, and intracerebral administration.

9. The vaccine composition of claim 7, wherein the vaccine composition further comprises a pharmaceutically acceptable excipient, diluent, or carrier.

10. A method for preventing or treating DENV infection, comprising administering the vaccine composition of claim 7 to a subject in need thereof.

11. The method of claim 10, wherein the DENV infection is at least one selected from the group consisting of Dengue Fever (DF), Dengue Hemorrhagic Fever (DHF), Dengue Shock Syndrome (DSS), and severe dengue.


SEQUENCE LISTING (14 sequences)

SEQ ID NO: 1 - DNA, 18 bp, synthetic construct
  source 1..18: mol_type = other DNA
caaacagcaggaccttgg

SEQ ID NO: 2 - DNA, 18 bp, synthetic construct
  source 1..18: mol_type = other DNA
atccatcctcacctctgt

SEQ ID NO: 3 - DNA, 57 bp, synthetic construct
  source 1..57: mol_type = other DNA
ggtaccctcgagatggactacaaagacgatgacgacaaggcagcaatcctggcatac

SEQ ID NO: 4 - DNA, 61 bp, synthetic construct
  source 1..61: mol_type = other DNA
ggaattccagatcctcttctgagatgagtttttgttcacgcgttcctttcttgaaccagtt

SEQ ID NO: 5 - DNA, 36 bp, synthetic construct
  source 1..36: mol_type = other DNA
ggaattcagcagcggcacgaccgcgtccacctcgca

SEQ ID NO: 6 - DNA, 31 bp, synthetic construct
  source 1..31: mol_type = other DNA
cgtctagattagctttcattatcactgtctc

SEQ ID NO: 7 - DNA, 93 bp, synthetic construct
  source 1..93: mol_type = other DNA
atggcagcaatcctggcatacaccataggaacgacacatttccaaagagtcctgattttcatcctactgacagctgtcgctccttcaatgaca

SEQ ID NO: 8 - DNA, 1185 bp, synthetic construct
  source 1..1185: mol_type = other DNA
atgcgctgtataggaatagcaaatagggacttcgtggaaggggtttcaggaggaagctgggttgacatagtcttagaacacggaagctgtgtgacgacgatggcaaaaaacaaaccaacactggactttgaactgataaaaacagaagccaaacaacccgccactctaaggaagtactgcatagaggctaagctgaccaacacgacaacagattcgcgctgcccaacacaaggggaacctagcctgaatgaagagcaggacaaaaggtttgtctgcaaacattctatggtagacagaggatggggaaatggatgcggattgtttggaaaaggaggcatcgtgacctgtgctatgttcacatgcaaaaagaatatgaaaggaaaaattgtgcagccagaaaacttggaatacaccatcgtgataacacctcactcaggggaagaacatgcagtcggaaataacacaggaaaacatggcaaggaagtcaaaataacaccacagagctccatcacagaagcggaactgacaggctatggcactgttacgatggagtgctctccgagaacgggcctcgacttcaatgagatggtgttgctgcaaatggaagataaagcttggctggtgcacagacaatggttcctagacctgccgttaccatggctgcccggagcagatacacaaggatcaaattggatacagaaagagacattggtcactttcaaaaatccccatgcaaaaaaacaggatgttgttgtcttaggatcccaagagggggccatgcacacagcactcacaggggctacggaaatccagatgtcatcaggaaacctactgttcacaggtcatcttaagtgcaggctgagaatggacaaactacagcttaaagggatgtcatactctatgtgtacagggaagtttaaagtcgtgaaggaaatagcagaaacacaacatggaacaatagtcattagagtacaatatgaaggagacggctctccatgcaagatcccttttgagataatggatctggaaaaaagacatgtcttaggtcgtctgattacagtcaacccaattgtaacagaaaaggacaggccagtcaacatagaagcagaacctccattcggagacagctacattatcataggagtggagccgggacaactgaagcttaactggttcaagaaagga

SEQ ID NO: 9 - DNA, 30 bp, synthetic construct
  source 1..30: mol_type = other DNA
gaacaaaaactcatctcagaagaggatctg

SEQ ID NO: 10 - DNA, 549 bp, synthetic construct
  source 1..549: mol_type = other DNA
atgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagc

SEQ ID NO: 11 - protein, 31 aa, synthetic construct
MAAILAYTIGTTHFQRVLIFILLTAVAPSMT

SEQ ID NO: 12 - protein, 395 aa, synthetic construct
MRCIGIANRDFVEGVSGGSWVDIVLEHGSCVTTMAKNKPTLDFELIKTEAKQPATLRKYCIEAKLTNTTTDSRCPTQGEPSLNEEQDKRFVCKHSMVDRGWGNGCGLFGKGGIVTCAMFTCKKNMKGKIVQPENLEYTIVITPHSGEEHAVGNNTGKHGKEVKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDRPVNIEAEPPFGDSYIIIGVEPGQLKLNWFKKG

SEQ ID NO: 13 - protein, 10 aa, synthetic construct
EQKLISEEDL

SEQ ID NO: 14 - protein, 183 aa, synthetic construct
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES